(19) United States
(12) Patent Application Publication
(10) Pub. No.: US 2024/0382737 A1
(43) Pub. Date: Nov. 21, 2024

(54) CONNECTOR COUPLING ASSEMBLY WITH REMOVABLE AND REPLACEABLE INTEGRATED LUER CONNECTOR

(71) Applicant: CareFusion 303, Inc., San Diego, CA (US)

(72) Inventors: Jason Andrew WINE, Brea, CA (US); George MANSOUR, Diamond Bar, CA (US)

(21) Appl. No.: 18/616,686

(22) Filed: Mar. 26, 2024

Related U.S. Application Data
(60) Provisional application No. 63/502,264, filed on May 15, 2023.

(51) Int. Cl. A61M 39/10; A61M 39/22

(52) U.S. Cl. CPC A61M 39/10; A61M 39/22; A61M 2039/1088

(57) ABSTRACT

A connector assembly including a body portion defining a cavity and a luer portion extending in the cavity, a proximal connector disposed at least partially in the cavity of the body portion, a distal connector slidably coupled to the body portion, and a compressible valve member mounted in a cavity of the proximal connector. The distal connector including first and second arms hingedly or pivotably coupled to each other. The valve member may have an internal chamber and a slit extending from a distal end portion of the valve member into the internal chamber. When the distal connector is coupled to a mating connector, the valve member is compressed and the slit is opened such that a fluid path extending from the inlet port through the internal chamber to the outlet port of the luer portion is opened to fluidly communicate the proximal connector with the mating connector.

### CROSS REFERENCE TO RELATED APPLICATION

This application claims the benefit of priority under 35 U.S.C. § 119 to U.S. Provisional Patent Application Ser. No. 63/502,264, entitled “CONNECTOR COUPLING ASSEMBLY WITH REMOVABLE AND REPLACEABLE INTEGRATED LUER CONNECTOR,” filed on May 15, 2023, the entire contents of which is incorporated herein by reference.

### FIELD OF THE INVENTION

The present disclosure generally relates to connectors, and, in particular, to connector couplings.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag. Often, tubing or catheters are coupled or secured to each other to allow fluid communication between various portions of tubing or catheters.

In some applications, such tubing or catheters may become dislodged due to improper securement and/or when the coupling is subject to forces greater than what the coupling is designed to withstand.

### SUMMARY

The present disclosure provides, in at least some embodiments, connector assembly, comprising a body portion having an inner surface defining a cavity terminating in an open end of the body portion, and a luer portion extending in the cavity and through the open end, a proximal connector disposed at least partially in the cavity of the body portion, and defining an inlet port and having an inner surface defining a cavity of the proximal connector, a distal connector slidably coupled to the body portion and comprising first and second arms hingedly or pivotably coupled to the body and defining (i) an outer surface of the distal connector, and (ii) an inner surface of the distal connector which defines a cavity terminating in an open end of the distal connector; and a compressible valve member mounted in the cavity of the proximal connector and comprising an internal chamber and a slit extending from a distal end portion of the compressible valve member into the internal chamber, wherein when the distal connector is coupled to a mating connector, the compressible valve member is compressed and the slit is opened such that a fluid path extending from the inlet port through the internal chamber to an outlet port of the luer portion is opened to fluidly communicate the proximal connector with the mating connector.

In some embodiments, the present disclosure provides a connector assembly comprising a body portion having an inner surface defining a cavity terminating in an open end of the body portion, and a luer portion extending in the cavity and through the open end, a proximal connector disposed at least partially in the cavity of the body portion, and defining an inlet port and having an inner surface defining a cavity of the proximal connector, wherein a fluid path extends from the inlet port through the luer portion to an outlet port of the luer portion, a distal connector slidably coupled to the body portion and configured to couple to a mating connector, and a compressible valve member mounted in the cavity of the proximal connector and comprising an internal chamber and a slit extending from a distal end of the compressible valve member into the internal chamber, and a post coupled to and extending longitudinally in the fluid path from the compressible valve member toward the outlet port of the luer portion, wherein when the distal connector is coupled to the mating connector the post is displaced away from the outlet port of the luer portion to compress the compressible valve member, open the slit, and permit flow through the fluid path and into the mating connector via the outlet port, and wherein when a force applied to the proximal connector exceeds a predetermined threshold, a spring member expands and exerts a force on the post to displace the post towards the outlet port of the luer portion to seal the outlet port.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 is a perspective view of an extension set including a connector assembly, in accordance with some embodiments of the present disclosure.

FIG. 2 illustrates a cross-sectional view of a connector assembly coupled with a mating needleless connector, in accordance with some embodiments of the present disclosure.

FIG. 3 illustrates a partial perspective view of the connector assembly of FIG. 2, in accordance with some embodiments of the present disclosure.

FIG. 4 illustrates an operational view of the connector assembly decoupled from the mating needleless connector due to pivoting open of a distal connector of the connector assembly when the connector assembly is subject to a proximal-direction force above a predetermined threshold, in accordance with some embodiments of the present disclosure.

FIG. 5 illustrates a cross-sectional operational view of a proximal connector of the connector assembly decoupled from the connector assembly for swabbing, in accordance with some embodiments of the present disclosure.

FIG. 6 illustrates a cross-sectional operational view of the proximal connector of the connector assembly coupled to the rest of the connector assembly after swabbing, in accordance with some embodiments of the present disclosure.

FIG. 7 is an elevation view of a detachable connector assembly, in accordance with some embodiments of the present disclosure.

FIG. 8 illustrates a cross-sectional view of the connector assembly of FIG. 7, in accordance with some embodiments of the present disclosure.

FIG. 9 is a detail view of the connector assembly of FIG. 8, in accordance with some embodiments of the present disclosure.

FIG. 10 is a cross-sectional view of an arm for a connector assembly, in accordance with some embodiments of the present disclosure.

FIG. 11 is a cross-sectional view of the arm of FIG. 10, in accordance with some embodiments of the present disclosure.

### DETAILED DESCRIPTION

The disclosed connector assembly incorporates a body portion having an inner surface defining a cavity terminating in an open end of the body portion and including a plurality of wings, a proximal connector disposed at least partially in the cavity and configured to couple to a medical fluid source, and a distal connector slidably coupled to the body portion via the plurality of wings and configured to couple to a mating needleless connector. The inner surface of the body portion may include a plurality of notches recessed therein. In some embodiments, the plurality of wings may extend from an outer surface of the proximal connector to an outer surface of the distal connector. The body portion may further include a luer portion extending in the cavity and through the open end thereof.

The proximal connector may define an inlet port and may have an inner surface defining a cavity of the proximal connector. A fluid path may extend from the inlet port through the luer portion to an outlet port of the luer portion. The proximal connector may further include a compressible valve member mounted therein. The compressible valve member may have an internal chamber and a slit extending from a distal end portion of the compressible valve member into the internal chamber. A post may extend longitudinally in the fluid path from the distal end portion of the compressible valve member toward the outlet port of the luer portion. A distal end of the post may extend into the luer portion. The distal connector may include first and second arms pivotably or hingedly coupled to each other and defining an outer surface of the distal connector. Each of the first and second arms may include at least one detent extending radially outward from the outer surface of the distal connector for engaging a corresponding notch of the plurality of notches of the body portion.

When the force applied to the proximal connector exceeds a predetermined threshold (e.g., a high pullout force), the detents of the first and second arms may disengage from the notches of the body portion to allow the distal connector to translate distally relative to the proximal connector and pivot the first and second arms radially outward to decouple the mating connector from the distal connector. Upon disengagement of the detents from the notches, the compressed valve member may expand like a spring and correspondingly move the post distally. Although a compressible valve member is described in the present disclosure as having features configured to move the post, it should be understood that the present disclosure also contemplates embodiments in which a spring member or other biasing device is used to move or bias the post. In some embodiments of the present disclosure a spring member can be used with a valve together to provide features for permitting or obstructing a fluid flow, and to move or bias the post.

As the post moves distally, protrusions on arms of the post may exert a distal force on the corresponding first and second arms of the distal connector, thereby causing the distal connector to move or translate distally. In particular, as the compressible valve member expands, each of the wings may slide along a slot of each of the first and second arms to allow the distal connector to translate distally relative to the proximal connector. As the compressible valve member continues to expand, a ramp surface of each of the slots may push against ramp portions of the wings to pivot the first and second arms of the distal connector radially outward and widen the opening at the open end of the distal connector. As the compressible valve member continues to expand distally, the compressible valve member may continue to exert a force on the post which moves or otherwise displaces the closed end of the post towards the outlet port of the luer portion to seal the outlet port.

As the first and second arms continue to pivot radially outward, the mating connector may then be released and completely decoupled from the distal connector. In this state, the outlet port of the luer portion may be sealed by a seal at the closed end of the post, thereby closing the fluid path and advantageously preventing microbial ingress into the fluid path. Further advantageously, the sealed outlet port may prevent the medical fluid, e.g., IV fluid from further exiting or otherwise spilling out of the fluid path via the outlet port of the luer portion upon the disconnection of the mating connector. Since the first and second arms are advantageously designed to release the mating needleless connector when a pull force exceeding the threshold pull force is applied. As such, both the connector assembly (for example, but not limited to a Texium valve) and the mating connector (for example, but not limited to a Smartsite valve) may automatically shut off at separation thereby preventing leakage or spillage of medical fluids upon accidental disconnection by higher pullout forces exceeding the predetermined threshold force. The proximal connector may be detachably coupled to the rest of the connector assembly (i.e., the body portion and distal connector assembly) to advantageously allow swabbing or otherwise disinfection of the luer portion of the proximal connector. The rest of the connector assembly (i.e., the body portion and distal connector assembly) may then be discarded and replaced with a new sterile body portion and distal connector assembly. The mating connector, when detached or decoupled from the connector assembly may also be swabbed or otherwise disinfected before being reattached to the connector assembly.

After swabbing, the mating connector may then be reconnected to the new sterile connector assembly. As the mating connector is reconnected to the distal connector the luer portion of the body portion may advance into the interior of the mating connector and a top surface of the mating connector may exert a force to urge the plurality of arms of the post proximally, which in turn may displace the closed end of the post away from the outlet port of the luer portion of the body portion, compress the compressible valve member such that the slit of the valve member opens. In the open position of the slit, the closed end of post is displaced from the outlet port of the luer portion, thereby the open slit re-opens the fluid path, and permits the medical fluid to flow through the outlet port of the luer portion and into the mating connector via a lumen and passage of the post. Accordingly, administration of the IV fluid from the fluid line to the catheter via the new sterile connector assembly may resume.

According to various embodiments of the present disclosure, when subject to lower pullout forces, e.g., forces below or equal to the predetermined proximal threshold (pullout) force, the connector assembly may be configured so as to advantageously retain the mating connector within the distal connector such that the fluid path remains open and medical fluid, e.g., IV fluid may be delivered from a fluid line into the catheter for administration to a patient.

The connector assembly of the various embodiments described herein-by releasing or otherwise decoupling from the mating connector, versus conventional couplers and related infusion systems in which the pullout force exceeding the predetermined threshold would result in dislodgement or decoupling of the actual catheter-is thereby advantageous in preventing the dislodgment of the catheter, potential resulting bleeding, medication leaks, as well as excessive ringing of alarms to alert a clinician of the dislodgement.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for case of understanding. Reference numbers may have letter suffixes appended to indicate separate instances of a common element while being referred to generically by the same number without a suffix letter.

While the following description is directed to the connection of medical fittings for the administration of medical fluid using the disclosed connector assembly, it is to be understood that this description is only an example of usage and does not limit the scope of the claims. Various aspects of the disclosed coupler may be used in any application where it is desirable to secure the connection of various tubing and fittings.

The disclosed connector assembly overcomes several challenges discovered with respect to certain conventional couplers utilized during PIVC blood draw. One challenge with certain conventional couplers is that certain conventional couplers may be improperly secured. Further, during use, certain conventional couplers may be designed to release or dislodge in response to relatively low pullout forces. For example, certain conventional couplers may release in response to pullout forces experienced during patients rolling over in bed, patients catching tubing or lines on bed rails, moving patients to a different bed, fidgeting by pediatric patients, and/or disoriented adult patients pulling out their lines. Indeed, the Association for Vascular Access (AVA) Annual Scientific Meeting in 2017 reported a 10% dislodgement rate for 1,000 patients fitted with peripheral IV catheters, translating to approximately 33 million dislodgements per year in the U.S. alone. Because the accidental or unintentional dislodgement of tubing, catheters, or fittings may interrupt the administration of medical fluids, the use of certain conventional couplers is undesirable.

Therefore, in accordance with the present disclosure, it is advantageous to provide connector assemblies as described herein that allow for improved securement of fittings or connectors. The disclosed connector assemblies are structured as described herein so as to permit the secure retention of the connectors, while allowing intentional removal of the connector as required.

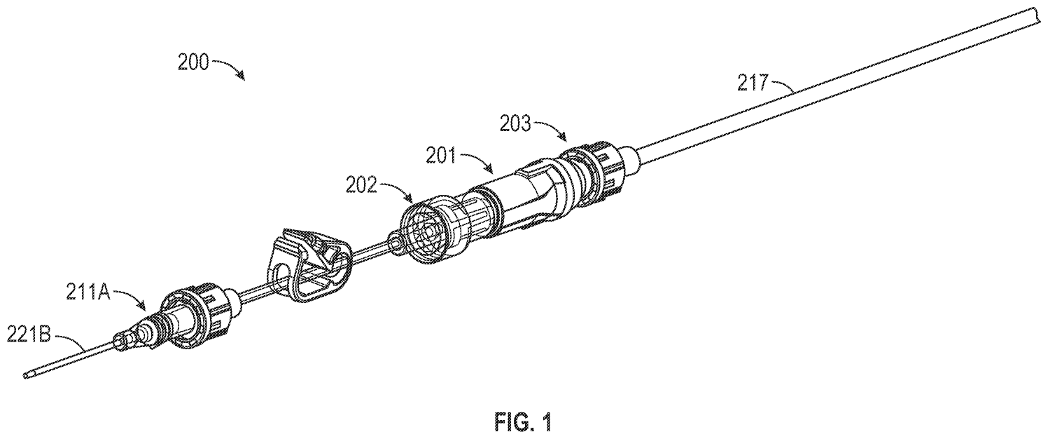

FIG. 1 is a perspective view of an extension set including a connector assembly, in accordance with some embodiments of the present disclosure. In accordance with various embodiments of the present disclosure, the extension set 200 may include a tubing 217 configured to couple to a medical fluid (e.g., IV fluid) source (not shown). As depicted, the extension set 200 may further include a detachable connector assembly 201 for selectively delivering the medical fluid to a catheter assembly 221A via a mating connector 202 (e.g., a needleless connector). The extension set 200 may further include a mating luer 203 (e.g., a male luer connector) for coupling the connector assembly 201 to the tubing 217. As shall be described in further detail below, a fluid path 238 may be defined at least partially from the lumen of the tubing 217, through a lumen of the mating luer 203, the connector assembly 201, and the mating connector 202. The medical fluid may thus be delivered to a catheter 221B of the catheter assembly 221A via the fluid path 238.

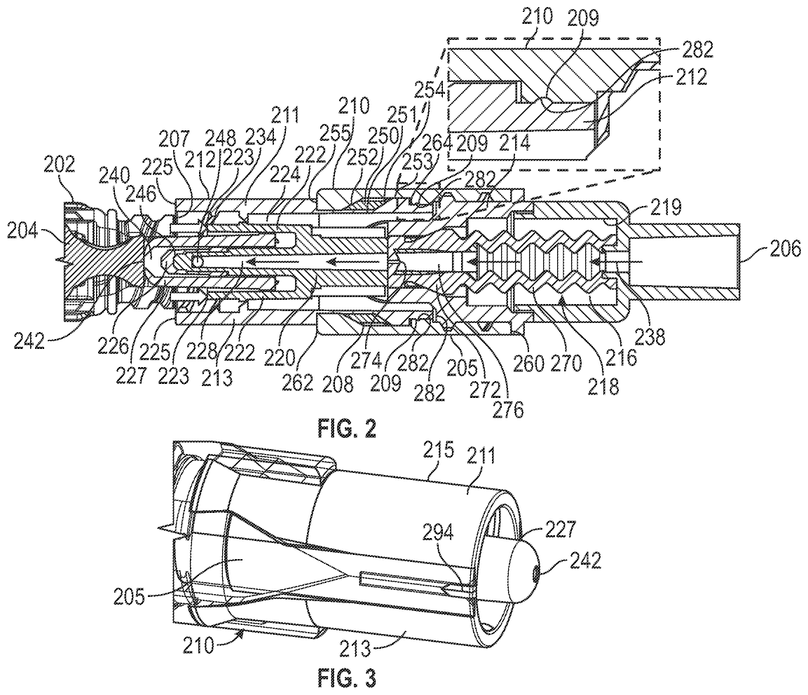

FIG. 2 illustrates a cross-sectional view of a connector assembly coupled with a mating needleless connector, in accordance with some embodiments of the present disclosure. FIG. 3 illustrates a partial perspective view of the connector assembly of FIG. 2, in accordance with some embodiments of the present disclosure. As illustrated in FIGS. 2 and 3, with continued reference to FIG. 1, the connector assembly 201 may include a body portion 205, a proximal connector 214 disposed at least partially in the body portion 205, and a distal connector 212 slidably coupled to the body portion 205. The body portion 205 may have an inner surface defining a cavity 208 terminating in an open end 262 of the body portion 205, and a luer portion 227 extending in the cavity 208 and through the open end 262 of the body portion 205. The inner surface of the body portion 205 may include a plurality of notches 282 recessed therein. In some embodiments, the body portion 205 may include a plurality of wings 210. In some embodiments, the plurality of wings 210 may extend from an outer surface of the proximal connector 214 to an outer surface 215 of the distal connector 212. As shall be described in further detail below with respect to the operation of the connector assembly 201, the distal connector 212 may be slidably coupled to the proximal connector 214 via the plurality of wings 210. In some embodiments, each of the plurality of wings 210 may have a proximal end 260 and a distal end forming the open end 262. Each of the wings 210 may have an inner surface 264 including a first linear portion 266 extending proximally from the open end 262, a ramp portion 252 extending proximally and radially outward from the first linear portion 266, and a second linear portion 270 extending proximally from the ramp portion 252.

In some embodiments, the proximal connector 214 may be disposed at least partially in the cavity 208 of the body portion 205. The proximal connector 214 may define an inlet port 206 and may have an inner surface 219 defining a cavity 216 of the proximal connector 214. A compressible valve member 218 may be mounted in the cavity 216 of the proximal connector 214. The compressible valve member 218 may include an internal chamber 276 and a slit 274 extending from a distal end portion of the compressible valve member 218 into the internal chamber 276. For example, in some embodiments the compressible valve member 218 may be a split-septum type of compressible valve member.

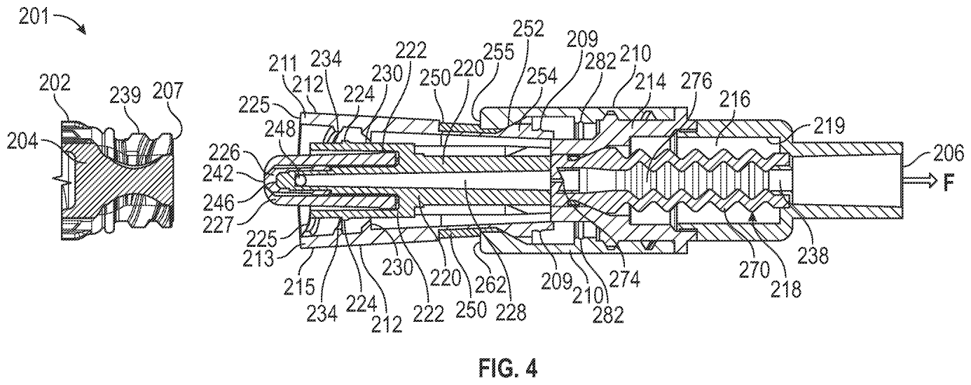

In some embodiments, the fluid path 238 may extend from the inlet port 206 through the luer portion 227, to an outlet port 242 of the luer portion 227. A post 220 may be disposed in the luer portion 227 extending longitudinally in the fluid path 238 from the proximal end portion of the compressible valve member 218 toward the outlet port 242 of the luer portion 227. In some embodiments, the post 220 may define a lumen 228 and may have an open end 244, an opposing closed end 246, and a passage 248 through a sidewall between the open end 244 and the closed end 246. In some embodiments, the post 220 may include a seal 226 on the outside surface of the closed end 246 to prevent fluid flow through the outlet port 242 of the luer portion 227. As depicted, the post 220 may have a plurality of arms 222 extending from the post 220 and along an exterior of the luer portion 227 toward the outlet port 242. The post 220 may further include a pair of protrusions 224 (illustrated in FIG. 4), one protrusion 224 of the pair of protrusions extending radially outward from an outer surface of each of the respective arms 222 of the post 140.

According to various embodiments, the distal connector 212 may have first and second arms 211 and 213 that are pivotable relative to each other (illustrated in FIG. 4). For example, in some embodiments, the first and second arms 211 and 213 may each include a pair of hinge portions having coupling apertures. The hinge portions and respective coupling apertures of the first and second arms 211 and 213 may be aligned coaxially such that pivot pins may be inserted into the aligned coupling apertures at opposing sides of the first and second arms 211 and 213. Accordingly, the pair of hinge portions of the first arm 211 may be rotatably coupled to the pair of hinge portions of the second arm 213, thereby pivotably coupling the first and second arms 211 and 213 of the distal connector 212 to each other.

In some embodiments of the present disclosure, the ability to pivot the first and second arms 211 and 213 is provided by the body portion 205 having a pivot bore or a pivot pin, and the first and second arms 211 and 213 having the other of the pivot bore or the pivot pin. The pivot bore can be located on opposite sides of the body 205, and each of the first and second arms 211 and 213 can have a pair of pivot pins configured to be received into the pivot bore.

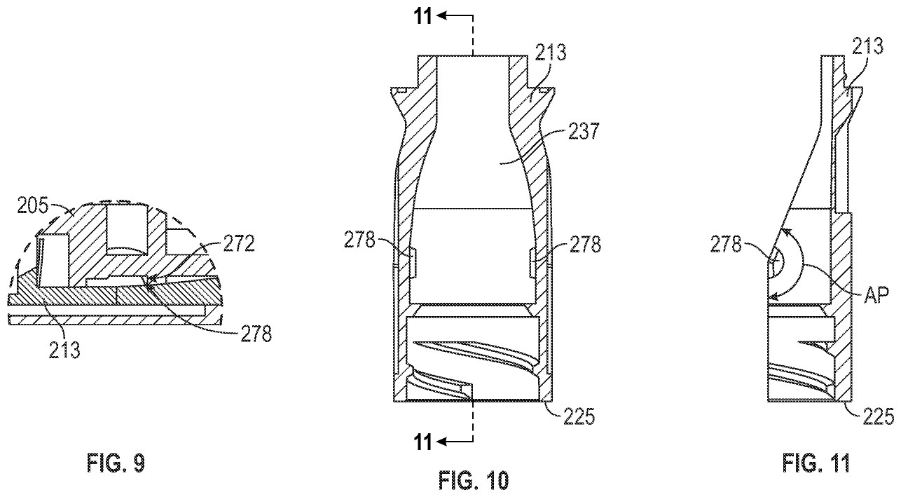

Referring to FIGS. 8 and 9, which show a cross-sectional view of the connector assembly 201 in the direction A-A and a detail view thereof, the body portion 205 includes a pivot bore 272 and each of the first and second arms 211 and 213 include a pivot pin 278 configured to be positioned within the pivot bore 272.

The second arm 213 is shown in FIG. 10 and shows an inner surface 237 of the distal connector 212 which defines a cavity 243 terminating in an open end 225 of the distal connector. The second arm 213 includes first and second pivot pins 278 each located along lateral side portions of the second arm such that the second arm 213 is pivotable about an axis that extends between the first and second pivot pins 278. In some aspects of the present disclosure, the first and second pivot pins 278 are located diametrically opposite to each other. Although the second arm 213 is shown in isolation for clarity, it should be understood that the present disclosure contemplates that the first arm 211 can have the same or similar features as the second arm 213.

A cross-sectional view of the second arm 213 of FIG. 10 is shown in FIG. 11 along the line C-C, which illustrates that in some embodiments, the pivot pins 278 can be configured with a partial circular cross-section. The partial circular cross-section is configured such that a first pivot pin 278 of each of the first and second arms 211 and 213 can be inserted into a first pivot bore 272 of the body, and a second pivot pin 278 of each of the first and second arms 211 and 213 can be inserted into a second pivot bore 272 of the body, thereby enabling the first pivot pin 278 of the first and second arms 211 and 213 to be located in space of the first pivot bore 272, and the second pivot pin 278 of the first and second arms 211 and 213 to be located in space of the second pivot bore 272.

With a pivot pin 278 of each of the first and second arms 211 and 213 positioned in a pivot bore 272, a partial circular cross-section of each pivot pin 278 having a perimeter that is less than 180 can permit each of the first and second arms 211 and 213 to pivot relative to the body potion 205. In some embodiments of the present disclosure, each pivot pin has a circular perimeter that extends about an angle AP around an axial center of the pivot pin 278. In some embodiments, the angle AP is between approximately 10 degrees and approximately 180 degrees. In some embodiments, the angle AP is between approximately 90 degrees and approximately 175 degrees. In some embodiments, the angle AP is between approximately 120 degrees and approximately 160 degrees. In the embodiment shown in FIG. 11, the angle AP is 158 degrees.

In some embodiments of the present disclosure, proximal and distal ends of the first and second arms 211 and 213 are configured to move toward and away from the body portion 205 when any of the first or second arms 211 are 213 are pivoted. To permit the proximal and distal ends of the first and second arms 211 and 213 to move toward and away from the body portion 205, the pivot pins are located between the proximal and distal ends of the first and second arms 211 and 213. Movement of the proximal and distal ends of the first and second arms 211 and 213 toward and away from the body portion 205 can also be permitted by the lateral sides of each of the first and second arms 211 and 213 having a chamfer at the proximal end portion thereof. In another aspect, when the first and second arms 211 and 213 are coupled to the pivot bore, a space is created between the first and second arms 211 and 213, from the pivot pin toward the proximal ends of the first and second arms 211 and 213.

When the distal connector 212 moves in a distal direction, relative to the proximal connector 214, the first and second arms 211 and 213 pivot so that the distal ends of the first and second arms 211 and 213 are moved radially outward. When the distal ends of the first and second arms 211 and 213 are moved radially outward, the mating connector 202 can be decoupled from the distal connector.

As illustrated, the first and second arms 211 and 213 may define (i) the outer surface 215 of the distal connector 212, and (ii) an inner surface 237 of the distal connector 212 which defines a cavity 243 terminating in an open end 225 of the distal connector 212. The inner surface 237 of the distal connector 212 may have at least one stop 230 (illustrated in FIG. 4) extending radially inward from the inner surface 221. As depicted, when the mating connector 202 is coupled to the distal connector 212, each protrusion 224 may abut a corresponding one of the at least one stops 230 to prevent unintended expansion of the compressible valve member 218 and distal movement of the post 220.

As further depicted, each of the first and second arms 211 and 213 may include at least one detent 209 extending radially outward from the outer surface 215 of the distal connector 212. When the distal connector 212 is coupled to the mating connector 202, the detents 209 of the first and second arms 211 and 213 engage the plurality of notches 282 to prevent decoupling of the mating connector 202 from the distal connector 212 when a force F applied to the proximal connector 214 (i.e., a proximal-direction force) is less than or equal to a predetermined proximal threshold (pullout) force.

In some embodiments, the outer surface 215 defined by the first and second arms 211 and 213 of the distal connector 212 may have a slot 250 recessed at least partially therein. The slot 250 may have a linear surface 251 extending proximally from a distal end 255 of the slot 250, and a ramp surface 254 extending from the linear surface 251 to a proximal end 253 of the slot 250. As depicted, the ramp portion 252 of each wing 210 may be parallel to the ramp surface 254 of each slot 250 and the first linear portion 266 of each wing 210 may be parallel to the linear surface 251 of each slot 250. According to various embodiments of the present disclosure, when the force F applied to the proximal connector 214 exceeds the predetermined threshold, the detents 209 may disengage from the notches 282, causing the distal connector 212 to translate distally relative to the proximal connector 214. Upon disengagement of the detents 209 from the notches 282, the ramp surfaces 254 of the slots 250 may push against the ramp portions 252 of the wings 210 to pivot the first and second arms 211 and 213 radially outward and widen the open end 225 of the distal connector 212 to release the mating connector 202 (as illustrated in FIG. 4).

In operation, the connector assembly 201 may generally be coupled to the mating or reciprocal connector 202 at a distal end thereof and be coupled to the mating luer connector 203 at a proximal end thereof. In some embodiments, the mating or reciprocal connector 202 may be a needleless connector, and the mating luer connector 203 may be a male luer connector. For example, as depicted in FIG. 2, mating connector 202 may be inserted and coupled into the open end 225 of the distal connector 212, and the mating luer connector 203 may be inserted and coupled to the inlet port 206 of the proximal connector 214. In some embodiments, the mating connector 202 may include threads 239 for mating with complimentary threads 175 in the open end 125 of the distal connector 212. When the distal connector 212 is coupled to the mating connector 202, the at least one detent 209 engages the at least one notch 282 to prevent decoupling of the mating connector 202 from the distal connector 212 at or below a predetermined proximal threshold (pullout) force applied to the proximal connector 214.

FIG. 2 depicts the coupled configuration of the mating connector 202 and the distal connector 212 of the connector assembly 201 when the force F below or equal to the predetermined proximal threshold (pullout) force is applied to the proximal connector 214. For example, the connector assembly 201 may be subject to proximal-direction pullout forces as a result of patients rolling over in bed, patients catching tubing or lines on bed rails, moving patients to a different bed, fidgeting by pediatric patients, and/or disoriented adult patients tugging on their lines. According to various embodiments of the present disclosure, when subject to lower pullout forces, e.g., forces F below or equal to the predetermined proximal threshold (pullout) force, the connector assembly 201 may be configured so as to retain the mating connector 202 within the distal connector 212 such that the fluid path 238 remains open and medical fluid, e.g., IV fluid may be administered from the fluid line or tubing 217 (which may be fluidly connected to a fluid container, e.g., an IV bag (not shown)) into the catheter assembly 221A (illustrated in FIG. 1). In particular, as illustrated in FIG. 2, when the distal connector 212 is coupled to the mating connector 202, the closed end 246 of the post 220 (along with the attached seal 226) may be displaced away from the outlet port 242 of the luer portion 227 to permit flow through the fluid path 238 via the luer portion 227.

As depicted, in the coupled configuration of the mating connector 202 and the distal connector 212, the luer portion 227 may extend through a top surface 207 of the mating connector 202 into a mating luer of the mating connector 202 to displace a flexible valve 204 of the mating connector 202. Accordingly, when the mating connector 202 is coupled to the distal connector 212, the mating connector 202 may exert a force to urge the plurality of arms 222 of the post 220 proximally, which in turn may cause the post 220 to compress the compressible valve member 218 proximally and displace the closed end 246 of the post 220 away from the outlet port 242 of the luer portion 227. Due to the compression, the slit 274 at the distal end portion of the compressible valve member 218 may be opened, thereby fluidly communicating the internal chamber 276 of the compressible valve member 218 with the lumen 228 of the post 220 and permitting flow through the fluid path 238 into the mating connector 202 via the outlet port 242 of the luer portion 227. For example, as illustrated in FIG. 2, the plurality of arms 222 and post 220 may be forced away from the outlet port 242 of the luer portion 227 by the top surface 207. In the open position, the closed end 246 of post 220 is displaced from the outlet port 242 of the luer portion 227, thereby permitting the medical fluid to flow between the outlet port 242 of the luer portion 227 and the lumen 228 of the post 220 through the passage 248.

FIG. 4 illustrates an operational view of the connector assembly 201 decoupled from the mating connector 202 due to pivoting open of the distal connector 212 of the connector assembly 201 when the connector assembly 201 is subject to the proximal-direction force F above the predetermined threshold, in accordance with some embodiments of the present disclosure. According to various embodiments of the present disclosure, when subject to higher pullout forces, e.g., forces F exceeding the predetermined proximal threshold (pullout) force, the connector assembly 201 may be configured so as to release or otherwise decouple the mating connector 202 from within the distal connector 212 at which point the fluid path 238 may close and the medical fluid, e.g., IV fluid may be discontinued from entering the fluid line or tubing 217 (which may be fluidly connected to a fluid container, e.g., an IV bag (not shown)). In some embodiments, the predetermined proximal threshold (pullout) force) may be approximately 5 pounds (lbs.) For example, if the patient having the catheter 221A inserted into their skin walks away from the infusion pump or accidental pulls on the fluid line or tubing 217 and the force exceeds 5 lbs, the connector assembly 201 may automatically release or decouple from the mating connector 202, effectively closing the fluid path 238 connecting the patient and the infusion fluid source, as shall be described in further detail below. The connector assembly 201 of the various embodiments described herein-by releasing or otherwise decoupling from the mating connector 202 versus conventional couplers and related infusion systems in which the pullout force exceeding the predetermined threshold would result in dislodgement or decoupling of the actual catheter-is thereby advantageous in preventing the dislodgment of the catheter 221B, potential resulting bleeding, medication leaks, as well as excessive ringing of alarms to alert a clinician of the dislodgement.

In particular, as illustrated in FIG. 4, when the force applied to the proximal connector 214 exceeds the predetermined threshold, the detents 209 may disengage from the notches 282 to allow the distal connector 212 to translate distally relative to the proximal connector 214 and pivot the first and second arms 211 and 213 radially outward to decouple the mating connector 202 from the distal connector 212, as shall be described in further detail below. For example, in operation when the proximal pullout force F applied to the proximal connector 214 exceeds the predetermined threshold, the pullout force F may cause the detents 209 of the first and second arms 211 and 213 to disengage or otherwise be displaced from the notches 282 of the wings 210. Upon disengagement of the detents 209 from the notches 282, the compressed valve member 218 may expand and correspondingly move the post 220 distally. As the post 220 moves distally, the protrusion 224 on each of the plurality of arms 222 of the post 220 may exert a distal force on the corresponding stop 230 which it abuts, thereby causing the distal connector 212 to move or translate distally. In particular, as illustrated in FIG. 4, as the compressible valve member 218 expands, the first linear portion 266 of each of the wings 210 may slide along the linear surface 251 of the slot 250 to allow the distal connector 212 to translate distally relative to the proximal connector 214. The distal connector 212 may continue to translate distally as each of the wings 210 continue to slide along the linear surface 251 of each slot 250 up to a point where the ramp surface 254 of each slot 250 contacts and abuts the ramp portion 252 of each wing 210. As the compressible valve member 218 continues to expand, the ramp surface 254 of each slots 250 may push against the ramp portions 252 of the wings 210 to pivot the first and second arms 211 and 213 radially outward and widen the opening at the open end 225 of the distal connector 212. As the compressible valve member 218 continues to expand distally, the compressible valve member 218 may continue to exert a force on the post 220 which moves or otherwise displaces the closed end 246 of the post 220 towards the outlet port 242 of the luer portion 227 to seal the outlet port 242.

As depicted, as the first and second arms 211 and 213 continue to pivot radially outward, the mating connector 202 may then be released and completely decoupled from the distal connector 212. In this state, the outlet port 242 of the luer portion 227 is sealed by the seal 226 at the closed end 246 of the post 220, thereby closing the fluid path 238 and advantageously preventing microbial ingress into the fluid path 238. Further advantageously, the sealed outlet port 242 may prevent the medical fluid, e.g., IV fluid from further exiting or otherwise spilling out of the fluid path 238 via the outlet port 242 of the luer portion 227 upon the disconnection of the mating connector 202.

Accordingly, the first and second arms 211 and 213 are advantageously be designed to release the mating needleless connector 202 when a pullout force F exceeding the threshold pullout force is applied to the tubing 217 and the proximal connector 214. As such, both the connector assembly 201 (for example, but not limited to a Texium valve) and the mating connector 202 (for example, but not limited to a SmartSite valve) may automatically shut off at separation thereby preventing (i) microbial ingress into the fluid path 138, and (2) leakage or spillage of medical fluids upon accidental disconnection by higher pullout forces exceeding the predetermined threshold force. The aforementioned configuration is advantageous over currently existing catheter dislodgement devices or couplers which may or may not be generally adhesive based, and capable of only preventing catheter dislodgement at lower pullout forces. These currently existing catheter dislodgement devices or couplers are not capable of preventing catheter dislodgement at higher pullout forces, but instead may release in response to higher pullout forces (for example forces exceeding 5 lbs.) experienced during patients rolling over in bed, patients catching tubing or lines on bed rails, moving patients to a different bed, fidgeting by pediatric patients, and/or disoriented adult patients pulling out their lines.

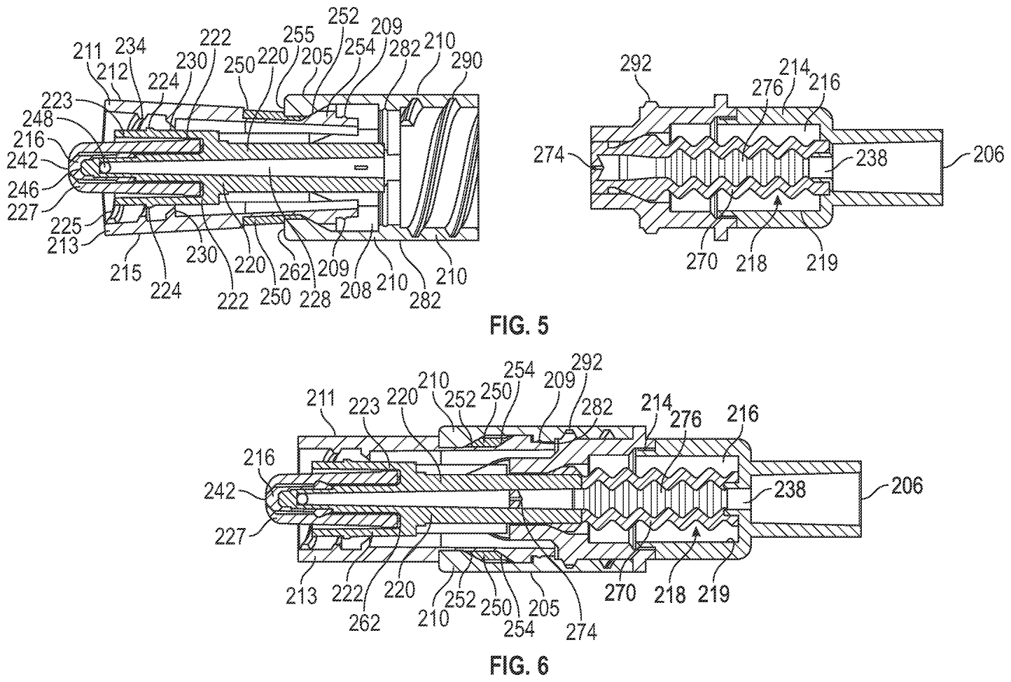

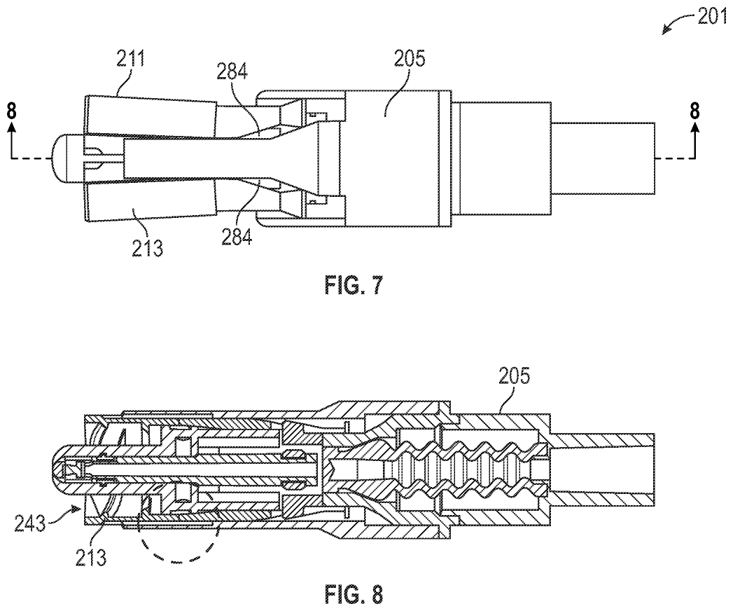

FIG. 5 illustrates a cross-sectional operational view of the proximal connector 214 of the connector assembly 201 decoupled from the connector assembly 201 for swabbing, in accordance with some embodiments of the present disclosure. FIG. 6 illustrates a cross-sectional operational view of the proximal connector 214 of the connector assembly coupled to the rest of the connector assembly 201 after swabbing, in accordance with some embodiments of the present disclosure. According to various embodiments of the present disclosure, the first and second arms 211 and 213 of the distal connector 212 may be returned from the pivoted state illustrated in FIG. 5 to the non-pivoted state illustrated in FIG. 6 by pinching together or otherwise exerting opposite-direction forces on the first and second arms 211 and 213 to pivot the first and second arms 211 and 213 radially inward to the original state prior to the radially-outward pivoting.

As illustrated in FIG. 5, the proximal connector 214 may be decoupled or otherwise detached from the rest of the connector assembly 201. For example, in the coupled or assembled configuration of the connector assembly 201, the proximal connector may be coupled in the cavity 208 of the body portion 205. As depicted, the inner surface of the body portion 205 may include a plurality of threads 290, and the outer surface of the proximal connector 214 may include complimentary threads 292 for engaging with the threads 290 of the body portion. In order to decouple or otherwise detach the proximal connector 214 from the rest of the connector assembly 201, the proximal connector 214 may be unthreaded or otherwise unscrewed and removed from the cavity 208 of the body portion 205. Although the decoupling of the proximal connector 214 from the rest of the connector assembly 201 is described herein with respect to a threaded engagement, the various embodiments are not limited to the aforementioned configuration, and the proximal connector 21 may instead be removably coupled to the body portion by any other suitable coupling or fastening method.

The aforementioned configuration in which the proximal connector 214 is removably coupled to the rest of the connector assembly is advantageous in that the luer portion of the proximal connector 214, one removed from the cavity 208 of the body portion 205, may then be swabbed or otherwise disinfected. In some embodiments, the rest of the connector assembly 201 (i.e., the body portion 205 and distal connector 212 assembly) may be discarded and replaced with a new and sterile body portion 205 and distal connector 212 assembly without breaching or otherwise infecting the fluid path 238. As illustrated in FIG. 6, the proximal connector 214 may then be attached or coupled to the new and sterile body portion 205 and distal connector 212 assembly.

Similarly, when decoupled or otherwise detached from the connector assembly 201, the mating connector 202 may also be swabbed or otherwise disinfected. The mating connector 202 may then be reconnected to the connector assembly 201 as illustrated in FIG. 2. For example, after disinfection of the mating connector 202, the mating connector 202 may then be coupled or connected to the new sterile connector assembly 201. The mating connector 202 may be advanced towards the luer portion 227 of the distal connector 212. As the mating connector 202 is advanced towards the luer portion 227 of the distal connector 212, the luer portion 227 may advance into the interior of the mating connector 202 and compress the valve member 204 of the mating connector 202. As the luer portion 227 advances further into the interior of the mating connector 202, the top surface 207 of the mating connector 202 may exert a force to urge the plurality of arms 222 of the post 220 proximally, which in turn may compress the compressible valve member 218 and displace the closed end 246 of the post 220 away from the outlet port 242 of the luer portion 227, as illustrated in FIG. 2. For example, as previously discussed the plurality of arms 222 and post 220 may be forced away from the outlet port 242 of the luer portion 227 by engagement of a distal end portion 223 of the arms 222 against the top surface 207 of the mating connector 202. In the open position, the closed end 246 of post 220 is displaced from the outlet port 242 of the luer portion 227, thereby re-opening the fluid path 238, and permitting the medical fluid to flow through the outlet port 242 of the luer portion 227 and into the mating connector 202 via the lumen 228 and the passage 248 of the post 220. Accordingly, administration of the IV fluid from the fluid line or tubing 217 to the catheter 221B via the new sterile connector assembly 201 may resume.

According to various embodiments of the present disclosure, when subject to lower pullout forces, e.g., forces below or equal to the predetermined proximal threshold (pullout) force, the connector assembly 201 may be configured so as to retain the mating connector 202 within the distal connector 212 such that the fluid path 238 remains open and medical fluid, e.g., IV fluid may be administered from the fluid line or tubing 217 into the catheter 221B. In particular, when the distal connector 212 is coupled to the mating connector 202, the closed end 246 of the post 220 (along with the attached seal 226) may be displaced away from the outlet port 242 of the luer portion 227 to permit flow through the fluid path 238 via the luer portion 227.

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1, a connector assembly, comprising: a body portion having an inner surface defining a cavity terminating in an open end of the body portion, and a luer portion extending in the cavity and through the open end; a proximal connector disposed at least partially in the cavity of the body portion, and defining an inlet port and having an inner surface defining a cavity of the proximal connector; a distal connector slidably coupled to the body portion and comprising: first and second arms hingedly or pivotably coupled to each other and/or the body and defining (i) an outer surface of the distal connector, and (ii) an inner surface of the distal connector which defines a cavity terminating in an open end of the distal connector; and a compressible valve member mounted in the cavity of the proximal connector and comprising an internal chamber and a slit extending from a distal end portion of the compressible valve member into the internal chamber, wherein when the distal connector is coupled to a mating connector, the compressible valve member is compressed and the slit is opened such that a fluid path extending from the inlet port through the internal chamber to an outlet port of the luer portion is opened to fluidly communicate the proximal connector with the mating connector.

Clause 2, the connector assembly of Clause 1, wherein: the inner surface of the body portion includes a plurality of notches recessed therein, and each of the first and second arms include at least one detent extending radially outward from the outer surface of the distal connector; and when the distal connector is coupled to a mating connector, the detents of the first and second arms engage the plurality of notches to prevent decoupling of the mating connector from the distal connector when a force applied to the proximal connector is less than or equal to a predetermined threshold.

Clause 3, the connector assembly of Clause 2, wherein when the force applied to the proximal connector exceeds the predetermined threshold, the detents of the first and second arms disengage from the plurality of notches to allow the distal connector to translate distally relative to the proximal connector and pivot the first and second arms radially outward to decouple the mating connector from the distal connector.

Clause 4, the connector assembly of any of Clauses 1 to 3, further comprising a post disposed in the luer portion and extending longitudinally in the fluid path from the distal end portion of the compressible valve member toward the outlet port of the luer portion, wherein when the distal connector is coupled to the mating connector a closed end of the post is displaced away from the outlet port of the luer portion to permit flow through the fluid path and into the mating connector via the outlet port.

Clause 5, the connector assembly of Clause 4, wherein the post comprises a plurality of arms extending from the post and along an exterior of the luer portion toward the outlet port, wherein when the mating connector is coupled to the distal connector, the mating connector exerts a force to urge the plurality of arms proximally, compress the compressible valve member, and displace the closed end of the post away from the outlet port of the luer portion.

Clause 6, the connector assembly of Clause 5, wherein when the distal connector is decoupled from the mating connector, a closed end of the post seals the outlet port of the luer portion to prevent fluid from exiting the fluid path via outlet port of the luer portion.

Clause 7, the connector assembly of Clause 6, wherein when the force applied to the proximal connector exceeds a predetermined threshold, the compressible valve member expands and exerts a force on the post to displace the closed end of the post towards the outlet port of the luer portion to seal the outlet port.

Clause 8, the connector assembly of any of Clauses 2 to 7, wherein an outer surface of each of the first and second arms of the distal connector comprises a slot recessed at least partially therein, the slot comprising a linear surface extending proximally from a distal end of the slot, and a ramp surface extending from the linear surface to a proximal end of the slot.

Clause 9, the connector assembly of Clause 8, wherein the body portion comprises a plurality of wings slidably coupling the distal connector to the proximal connector, each of the plurality of wings comprising a proximal end and a distal end, an inner surface including a first linear portion extending proximally from the distal end, a ramp portion extending proximally and radially outward from the first linear portion, and a second linear portion extending proximally from the ramp portion.

Clause 10, the connector assembly of Clause 9, wherein the ramp portion of each of the plurality of wings is parallel to the ramp surface of a corresponding slot of the first and second arms of the distal connector, and the first linear portion is parallel to the linear surface of the slot.

Clause 11, the connector assembly of Clause 10, wherein when a force exceeding a predetermined threshold is applied to the proximal connector and the detents of the first and second arms disengage from the plurality of notches, the first linear portion slides along the linear surface of the slot to allow the distal connector to translate distally relative to the proximal connector.

Clause 12, the connector assembly of Clause 11, wherein when the force exceeding the predetermined threshold is applied to the proximal connector and the detents of the first and second arms disengage from the plurality of notches causing the distal connector to translate distally relative to the proximal connector, the ramp surface of the slot pushes against the ramp portion of the plurality of wings wing to pivot the first and second arms radially outward and widen the open end of the distal connector to release the mating connector.

Clause 13, a connector assembly, comprising: a body portion having an inner surface defining a cavity terminating in an open end of the body portion, and a luer portion extending in the cavity and through the open end; a proximal connector disposed at least partially in the cavity of the body portion, and defining an inlet port and having an inner surface defining a cavity of the proximal connector, wherein a fluid path extends from the inlet port through the luer portion to an outlet port of the luer portion; a distal connector slidably coupled to the body portion and configured to couple to a mating connector; and a compressible valve member mounted in the cavity of the proximal connector and comprising an internal chamber and a slit extending from a distal end of the compressible valve member into the internal chamber; and a post coupled to and extending longitudinally in the fluid path from the compressible valve member toward the outlet port of the luer portion, wherein when the distal connector is coupled to the mating connector the post is displaced away from the outlet port of the luer portion to compress the compressible valve member, open the slit, and permit flow through the fluid path and into the mating connector via the outlet port, and wherein when a force applied to the proximal connector exceeds a predetermined threshold, the a spring member expands and exerts a force on the post to displace the post towards the outlet port of the luer portion to seal the outlet port.

Clause 14, the connector assembly of Clause 13, wherein the distal connector comprises first and second arms pivotably coupled to each other and/or the body and defining (i) an outer surface of the distal connector, and (ii) an inner surface of the distal connector which defines a cavity terminating in an open end of the distal connector.

Clause 15, the connector assembly of Clause 14, wherein: the inner surface of the body portion includes a plurality of notches recessed therein, and each of the first and second arms include at least one detent extending radially outward from the outer surface of the distal connector; and when the distal connector is coupled to the mating connector, the detents of the first and second arms engage the plurality of notches to prevent decoupling of the mating connector from the distal connector when a force applied to the proximal connector is less than or equal to a predetermined threshold.

Clause 16, the connector assembly of Clause 15, wherein when the force applied to the proximal connector exceeds the predetermined threshold, the detents of the first and second arms disengage from the plurality of notches to allow the distal connector to translate distally relative to the proximal connector and pivot the first and second arms radially outward to decouple the mating connector from the distal connector.

Clause 17, the connector assembly of Clause 15, wherein an outer surface of each of the first and second arms of the distal connector comprises a slot recessed at least partially therein, the slot comprising a linear surface extending proximally from a distal end of the slot, and a ramp surface extending from the linear surface to a proximal end of the slot.

Clause 18, the connector assembly of Clause 17, wherein the body portion comprises a plurality of wings slidably coupling the distal connector to the proximal connector, each of the plurality of wings comprising a proximal end and a distal end, an inner surface including a first linear portion extending proximally from the distal end, a ramp portion extending proximally and radially outward from the first linear portion, and a second linear portion extending proximally from the ramp portion.

Clause 19, the connector assembly of Clause 18, wherein the ramp portion of each of the plurality of wings is parallel to the ramp surface of a corresponding slot of the first and second arms of the distal connector, and the first linear portion is parallel to the linear surface of the slot.

Clause 20, the connector assembly of Clause 19, wherein when the force exceeding the predetermined threshold is applied to the proximal connector and the detents of the first and second arms disengage from the plurality of notches, the first linear portion slides along the linear surface of the slot to allow the distal connector to translate distally relative to the proximal connector.

Clause 21, the connector assembly of Clause 20, wherein when the force exceeding the predetermined threshold is applied to the proximal connector and the detents of the first and second arms disengage from the plurality of notches causing the distal connector to translate distally relative to the proximal connector, the ramp surface of the slot pushes against the ramp portion of the plurality of wings wing to pivot the first and second arms radially outward and widen the open end of the distal connector to release the mating connector.

Clause 22, the connector assembly of any of Clauses 13 to 21, wherein the post comprises a plurality of arms extending from the post and along an exterior of the luer portion toward the outlet port, and wherein when the mating connector is coupled to the distal connector, the mating connector exerts a force to urge the plurality of arms proximally, compress the compressible valve member, and displace the a closed end of the post away from the outlet port of the luer portion.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim clement is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A connector assembly, comprising:
a body portion having an inner surface defining a cavity terminating in an open end of the body portion, and a luer portion extending in the cavity and through the open end;
a proximal connector disposed at least partially in the cavity of the body portion, and defining an inlet port and having an inner surface defining a cavity of the proximal connector;
a distal connector slidably coupled to the body portion and comprising:
first and second arms pivotably coupled to the body portion and defining (i) an outer surface of the distal connector, and (ii) an inner surface of the distal connector which defines a cavity terminating in an open end of the distal connector; and
a compressible valve member mounted in the cavity of the proximal connector and comprising an internal chamber and a slit extending from a distal end portion of the compressible valve member into the internal chamber,
wherein when the distal connector is coupled to a mating connector, the compressible valve member is compressed and the slit is opened such that a fluid path extending from the inlet port through the internal chamber to an outlet port of the luer portion is opened to fluidly communicate the proximal connector with the mating connector.

2. The connector assembly of claim 1, wherein:
the inner surface of the body portion includes a plurality of notches recessed therein, and each of the first and second arms include at least one detent extending radially outward from the outer surface of the distal connector; and
when the distal connector is coupled to a mating connector, the detent of the first and second arms engage the plurality of notches to prevent decoupling of the mating connector from the distal connector when a force applied to the proximal connector is less than or equal to a predetermined threshold.

3. The connector assembly of claim 2, wherein when the force applied to the proximal connector exceeds the predetermined threshold, the detent of the first and second arms disengage from the plurality of notches to allow the distal connector to translate distally relative to the proximal connector and pivot the first and second arms radially outward to decouple the mating connector from the distal connector.

4. The connector assembly of claim 1, further comprising a post disposed in the luer portion and extending longitudinally in the fluid path from the distal end portion of the compressible valve member toward the outlet port of the luer portion, wherein when the distal connector is coupled to the mating connector a closed end of the post is displaced away from the outlet port of the luer portion to permit flow through the fluid path and into the mating connector via the outlet port.

5. The connector assembly of claim 4, wherein the post comprises a plurality of arms extending from the post and along an exterior of the luer portion toward the outlet port, wherein when the mating connector is coupled to the distal connector, the mating connector exerts a force to urge the plurality of arms proximally, compress the compressible valve member, and displace the closed end of the post away from the outlet port of the luer portion.

6. The connector assembly of claim 5, wherein when the distal connector is decoupled from the mating connector, a closed end of the post seals the outlet port of the luer portion to prevent fluid from exiting the fluid path via outlet port of the luer portion.

7. The connector assembly of claim 6, wherein when the force applied to the proximal connector exceeds a predetermined threshold, the compressible valve member expands and exerts a force on the post to displace the closed end of the post towards the outlet port of the luer portion to seal the outlet port.

8. The connector assembly of claim 2, wherein an outer surface of each of the first and second arms of the distal connector comprises a slot recessed at least partially therein, the slot comprising a linear surface extending proximally from a distal end of the slot, and a ramp surface extending from the linear surface to a proximal end of the slot.

9. The connector assembly of claim 8, wherein the body portion comprises a plurality of wings slidably coupling the distal connector to the proximal connector, each of the plurality of wings comprising a proximal end and a distal end, an inner surface including a first linear portion extending proximally from the distal end, a ramp portion extending proximally and radially outward from the first linear portion, and a second linear portion extending proximally from the ramp portion.

10. The connector assembly of claim 9, wherein the ramp portion of each of the plurality of wings is parallel to the ramp surface of a slot of the first and second arms of the distal connector, and the first linear portion is parallel to the linear surface of the slot.

11. The connector assembly of claim 10, wherein when a force exceeding a predetermined threshold is applied to the proximal connector and the detent of the first and second arms disengage from the plurality of notches, the first linear portion slides along the linear surface of the slot to allow the distal connector to translate distally relative to the proximal connector.

12. The connector assembly of claim 11, wherein when the force exceeding the predetermined threshold is applied to the proximal connector and the detent of the first and second arms disengage from the plurality of notches causing the distal connector to translate distally relative to the proximal connector, the ramp surface of the slot pushes against the ramp portion of the plurality of wings to pivot the first and second arms radially outward and widen the open end of the distal connector to release the mating connector.

13. A connector assembly, comprising:
a body portion having an inner surface defining a cavity terminating in an open end of the body portion, and a luer portion extending in the cavity and through the open end;
a proximal connector disposed at least partially in the cavity of the body portion, and defining an inlet port and having an inner surface defining a cavity of the proximal connector, wherein a fluid path extends from the inlet port through the luer portion to an outlet port of the luer portion;
a distal connector slidably coupled to the body portion and configured to couple to a mating connector; and
a compressible valve member mounted in the cavity of the proximal connector and comprising an internal chamber and a slit extending from a distal end of the compressible valve member into the internal chamber; and
a post coupled to and extending longitudinally in the fluid path from the compressible valve member toward the outlet port of the luer portion,
wherein when the distal connector is coupled to the mating connector the post is displaced away from the outlet port of the luer portion to compress the compressible valve member, open the slit, and permit flow through the fluid path and into the mating connector via the outlet port, and
wherein when a force applied to the proximal connector exceeds a predetermined threshold, a spring member expands and exerts a force on the post to displace the post towards the outlet port of the luer portion to seal the outlet port.

14. The connector assembly of claim 13, wherein the distal connector comprises first and second arms pivotably coupled to the body portion and defining (i) an outer surface of the distal connector, and (ii) an inner surface of the distal connector which defines a cavity terminating in an open end of the distal connector.

15. The connector assembly of claim 14, wherein:
the inner surface of the body portion includes a plurality of notches recessed therein, and each of the first and second arms include at least one detent extending radially outward from the outer surface of the distal connector; and
when the distal connector is coupled to the mating connector, the detent of the first and second arms engage the plurality of notches to prevent decoupling of the mating connector from the distal connector when a force applied to the proximal connector is less than or equal to a predetermined threshold.

16. The connector assembly of claim 15, wherein when the force applied to the proximal connector exceeds the predetermined threshold, the detent of the first and second arms disengage from the plurality of notches to allow the distal connector to translate distally relative to the proximal connector and pivot the first and second arms radially outward to decouple the mating connector from the distal connector.

17. The connector assembly of claim 15, wherein an outer surface of each of the first and second arms of the distal connector comprises a slot recessed at least partially therein, the slot comprising a linear surface extending proximally from a distal end of the slot, and a ramp surface extending from the linear surface to a proximal end of the slot.

18. The connector assembly of claim 17, wherein the body portion comprises a plurality of wings slidably coupling the distal connector to the proximal connector, each of the plurality of wings comprising a proximal end and a distal end, an inner surface including a first linear portion extending proximally from the distal end, a ramp portion extending proximally and radially outward from the first linear portion, and a second linear portion extending proximally from the ramp portion.

19. The connector assembly of claim 18, wherein the ramp portion of each of the plurality of wings is parallel to the ramp surface of a slot of the first and second arms of the distal connector, and the first linear portion is parallel to the linear surface of the slot.

20. The connector assembly of claim 13, wherein the post comprises a plurality of arms extending from the post and along an exterior of the luer portion toward the outlet port, and wherein when the mating connector is coupled to the distal connector, the mating connector exerts a force to urge the plurality of arms proximally, compress the compressible valve member, and displace a closed end of the post away from the outlet port of the luer portion.
